(19) United States
(12) Patent Application Publication
(10) Pub. No.: US 2010/0292186 A1
(43) Pub. Date: Nov. 18, 2010

(54) Pharmaceutical Composition for Treatment or Prevention of Liver Cancer

(75) Inventors: Akimasa Nakao, Aichi (JP); Katsutoshi Sakakima, Aichi (JP)

Correspondence Address: MORGAN LEWIS & BOCKIUS LLP, 1111 PENNSYLVANIA AVENUE NW, WASHINGTON, DC, 20004, US

(73) Assignee: National University Corporation Nagoya University, Nagoya-Shi, Aichi (JP)

(21) Appl. No.: 12/223,778

(22) PCT Filed: Feb. 6, 2007
(86) PCT No.: PCT/JP2007/052024

§ 371 Date: Mar. 29, 2010

(30) Foreign Application Priority Data
Feb. 10, 2006 (JP) 2006-034310

(51) Int. Cl. A61K 31/685; A61P 35/00

(52) U.S. Cl. 514/78

(57) ABSTRACT

An object of the present invention is to provide an excellent liver cancer treatment agent or prevention agent. The present invention provides a pharmaceutical composition for treating or preventing liver cancer, comprising menatetrenone, which is one species of vitamin K, and a phospholipid, said phospholipid being preferably egg yolk lecithin or soybean lecithin.

### TECHNICAL FIELD

The present invention relates to a pharmaceutical composition comprising vitamins and phospholipids, and more specifically, to a pharmaceutical composition for treating or preventing liver cancer, comprising menatetrenone (also referred to as “vitamin K2” or “MK-4”) which is one species of vitamin K, and a lecithin.

### BACKGROUND ART

So far, it has been reported (for instance, refer to Zhong-Qian Li et al., Life Sciences 70 (2002) 2085-2100, and Ziqiu Wang et al., Hepatology, 1995; 22: 876-882) that menatetrenone had an inhibitory effect on the growth of human hepatocellular cancer cell lines (for instance, Hep-3B, Hep-G2, Huh-7 and the like).

In addition, hepatocellular cancer patients suffer a high rate of portal venous invasion, and once portal venous invasion (hereinafter referred to as “PVI”) occurs, the prognostic of the foregoing is known to be extremely poor. Then, the high value of Des-γ-Carboxy Prothrombin (hereinafter referred to as “DCP”) in hepatocellular cancer patients is known to be related tightly to the progression of PVI thereafter (for instance, refer to Koike Y., et al Cancer 2001; 91: 561-569). Here, DCP is a prothrombin also referred to as PIVKA-II (Protein Induced by Vitamin K Absence or Antagonist), which does not have a normal coagulation activity and known to increase in a vitamin K-deprived situation, and is a protein used as a marker of vitamin K deprivation/vitamin K absorption defect, and used widely as a tumor marker in liver cancer.

In addition, it has been reported that by administrating vitamin K2 after treatment of hepatocellular cancer, the occurrence of PVI could be suppressed, and that the prognosis could be improved by suppressing the recurrence of hepatocellular cancer (for instance, refer to International Publication WO 03/105819 A1).

Meanwhile, although lecithins, which are a species of phospholipid, have been reported to have anticarcinogenic action (for instance, refer to Munder P G., et al Clinical Bulletin (Memorial Sloan-Kettering Cancer Center) (1976) Vol. 6, No. 2, pp. 80.), it has not been disclosed that the lecithins were effective on liver cancer.

In addition, although an injectable formulation comprising a liposoluble anticancer agent, a lecithin and another constituent have been reported (for instance, refer to Japanese Patent Application Laid-open No. H11-209307), it has not been disclosed that such an injectable formulation was effective on liver cancer.

Patent Document 1: International Publication WO 03/105819 A1

Patent Document 2: Japanese Patent Application Laid-open No. H11-209307

Non-Patent Document 1: Zhong-Qian Li et al., Life Sciences 70 (2002) 2085-2100

Non-Patent Document 2: Ziqiu Wang et al., Hepatology, 1995; 22: 876-88

Non-Patent Document 3: Koike Y., et al Cancer 2001; 91: 561-569

Non-Patent Document 4: Munder P G., et al Clinical Bulletin (Memorial Sloan-Kettering Cancer Center) (1976) Vol. 6, No. 2, pp. 80.

### DISCLOSURE OF INVENTION

### Problems to be Solved by the Invention

A better liver cancer treatment agent is desired from the point of view of effective treatment of liver cancer. Thus, an object of the present invention is to provide an excellent liver cancer treatment agent or prevention agent.

In view of the above situation, the present inventors engaged in earnest studies for a better liver cancer treatment agent, and discovered that, compared to menatetrenone alone, which is a species of vitamin K, a combination with a lecithin as a phospholipid promotes growth inhibition of liver cancer by menatetrenone, which is a species of vitamin K, and arrived at completing the present invention.

### Means for Solving the Problems

That is to say, the present invention provides:

[1] a pharmaceutical composition for treating or preventing liver cancer, comprising vitamin K and a phospholipid,
[2] the pharmaceutical composition as described in item [1], wherein the vitamin K is menatetrenone,
[3] the pharmaceutical composition as described in items [1] or [2], wherein the phospholipid is selected from the group consisting of egg yolk lecithin, soybean lecithin, hydrogenated lecithins of the foregoing, phosphatidylcholine, phosphatidylserine, phosphatidic acid, phosphatidyl inositol, phosphatidyl ethanolamine, sphingomyelin and lysophosphatidylcholine,
[4] the pharmaceutical composition as described in any one of items [1] to [3], wherein the phospholipid is egg yolk lecithin or soybean lecithin,
[5] the pharmaceutical composition as described in any one of items [1] to [4], wherein the mixing ratio of the menatetrenone and the phospholipid (menatetrenone/phospholipid) is a ratio of from 1/10 to 10/1 in mg/day/body weight,
[6] the pharmaceutical composition as described in any one of items [1] to [5], wherein the menatetrenone is administered at a dose of from 1.0 to 100 mg/day/body weight,
[7] the pharmaceutical composition as described in any one of items [1] to [6], wherein the phospholipid is administered at a dose of from 1.0 to 100 mg/day/body weight,
[8] the pharmaceutical composition as described in any one of items [1] to [7], which is administered orally,
[9] method for treating or preventing a liver cancer, comprising administering a pharmaceutical composition comprising vitamin K and a phospholipid, and
[10] use of vitamin K and phospholipid for preparing a liver cancer treatment or prevention pharmaceutical composition.

### ADVANTAGEOUS EFFECTS OF THE INVENTION

According to the present invention, compared to vitamin K alone, combination with a lecithin as a phospholipid promotes the growth inhibition of liver cancer by menatetrenone, providing a pharmaceutical composition as a better liver cancer treatment or prevention agent.

### BEST MODE FOR CARRYING OUT THE INVENTION

Hereinafter, the present invention will be described in more detail giving examples; however, the present invention is not limited to these.

The pharmaceutical composition according to the present invention comprises menatetrenone, which is a species of vitamin K, (also referred to as “vitamin K2” or “MK-4”) and a phospholipid, and has application for liver cancer treatment or prevention. Liver cancers that are the subjects of the present invention include, although not limited to the following, liver cancers carcinogenized from chronic hepatitis and hepatic cirrhosis, the hepatic cirrhosis including hepatitis C and hepatitis B due to hepatitis viruses.

Menatetrenone used in the present invention has the chemical name 2-methyl-3-tetraprenyl-1,4-naphthoquinone, and the structural formula thereof is shown below:

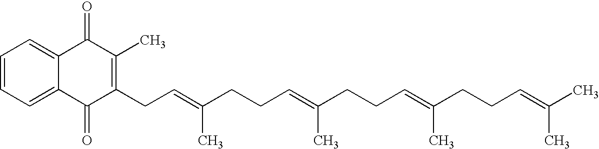

Menatetrenone is a yellow crystalline or oily substance, which is odorless and tasteless, and is prone to decomposition by light. In addition, it is almost insoluble in water, Menatetrenone, also referred to as vitamin K2, the pharmacological action thereof being one involved, in the process of protein synthesis of blood coagulation factors (prothrombin, VII, IX and X), in the carboxylation reaction during the conversion of a glutamic acid residue into γ-carboxy glutamic acid, which is biologically active, promotes hepatic synthesis of normal prothrombin and so on, activating the hemostatic mechanism of the organism for a physiological expression of hemostatic action.

Menatetrenone, the active ingredient of the pharmaceutical composition according to the present invention, may be an anhydrate and may also form a hydrate. In addition, crystal polymorphism may exist for menatetrenone, any of the crystal forms may be single or may be a mixture of crystal forms, without limitation.

Menatetrenone used in the present invention can be prepared by well known methods, and, in addition to being preparable readily according to methods disclosed in Japanese Patent Application Laid-open No. S49-55650 as a representative example, it can also be acquired readily from synthesis manufacturers. In addition, menatetrenone can also be acquired as formulations such as capsule and injectable.

Examples of phospholipid used in the present invention include egg yolk lecithin, soybean lecithin, hydrogenated lecithins of the foregoing, phosphatidylcholine, phosphatidylserine, phosphatidic acid, phosphatidyl inositol, phosphatidyl ethanolamine, lysophosphatidylcholine and the like, purified from natural products or semi-synthesized ones. Among these, for the phospholipid used in the present invention, egg yolk lecithin and soybean lecithin are preferred.

The pharmaceutical composition according to the present invention is a composition comprising menatetrenone and phospholipid, and the mixing ratio of the fore going (menatetrenone/phospholipid) is a ratio in mg/day/body weight of from 1/10 to 10/1 preferably of from 2/10 to 10/2, more preferably of from 4/10 to 10/4 and even more preferably of from 5/10 to 10/5.

As the pharmaceutical composition according to the present invention, menatetrenone and egg yolk lecithin or soybean lecithin may be used as-is, or, may be mixed with a constituent used generally as a raw material in pharmaceutical formulation, such as, a well known pharmacologically acceptable carrier (example: excipient, binder, disintegrant, lubricant, colorant, flavorant and, as necessary, stabilizer, emulsifier, absorption accelerator, surfactant, pH adjuster, antiseptic agent, antioxidant and the like), and formulated by methods used ordinarily. Furthermore, as necessary, constituents such as vitamins and amino acids may be mixed. Specific examples of excipient include lactose, corn starch, sucrose, fructose, sorbit, crystalline cellulose, and the like. Specific examples of binder include polyvinyl alcohol, polyvinyl ether, ethyl cellulose, methyl cellulose, gum arabic, thragacanth, gelatin, shellac, hydroxypropyl cellulose, polyvinylpyrrolidone, and the like. Specific examples of disintegrant include starch, agar, gelatin powder, crystalline cellulose, calcium carbonate, sodium bicarbonate, calcium citrate, dextrin, pectin, and the like. Specific examples of lubricant include magnesium stearate, talc, polyethyleneglycol, silica, hydrogenated vegetable oil, and the like. Specific examples of colorant include those that are allowed to be added to a pharmaceutical product. Specific examples of flavorant include cocoa powder, menthol, aromatic acid, peppermint oil, cinnamon powder, and the like.

In the present invention, although the administration mode of the pharmaceutical composition comprising menatetrenone and phospholipid is not limited in particular, administrating perorally is preferred. The pharmaceutical composition according to the present invention is a drug, which is constituted by drugs comprising menatetrenone alone and phospholipid alone, or which comprises both menatetrenone and phospholipid. Note that the drug comprising menatetrenone and phospholipid means a drug for administering, simultaneously or by leaving a fixed interval that is effective for the treatment, drugs obtained by formulating simultaneously the drugs alone, and a plurality of drugs obtained by formulating separately the drugs alone.

In order to formulate the pharmaceutical composition according to the present invention, it can be turned into a form such as tablet, powder, subtle granule, granule, capsule, syrup, suppository, injectable, ointment and skin patch by ordinary methods in the technical field of formulation. In addition, sugar coating, gelatin coating, and other suitable coatings as necessary may be performed on tablet, powder, subtle granule and granule. Note that, encapsulated formulation of menatetrenone can be acquired as product names Kaytwo capsule (manufactured by Eisai Co. Ltd.) and Glakay capsule (manufactured by Eisai Co. Ltd.), in addition, syrup can be acquired as product name Kaytwo syrup (manufactured by Eisai Co. Ltd.), and injectable can be acquired as product name Kaytwo N injectable (manufactured by Eisai Co. Ltd.).

The pharmaceutical composition according to the present invention is useful for treating or preventing liver cancer in mammals (for instance, human, mouse, rat, guinea pig, rabbit, dog, horse, monkey and the like), in particular, it is effective for treating or preventing liver cancer in human.

As the dose of menatetrenone in the pharmaceutical composition of the present invention, it is in general from 1.0 to 100 mg/day/body weight, preferably from 2.0 to 80 mg/day/body weight, more preferably from 5.0 to 60 mg/day/body weight. In addition, the dose of phospholipid in the pharmaceutical composition of the present invention is in general from 1.0 to 100 mg/day/body weight, preferably from 2.0 to 80 mg/day/body weight and more preferably from 5.0 to 60 mg/day/body weight.

### Examples

Hereinafter, experimental examples of the present invention will be given; however, these are illustrative, and the present invention is not limited to these experimental examples. Those skilled in the art can apply a variety of modifications not only to experimental examples shown hereinafter but also to the Claims in the present specification, and such modifications are also included in the scope of the present application.

### Examples

In the present invention, experiments were carried out by the following material and methods.

### A: On In Vitro Experiments

(1) Cell Lines and Culture Conditions

Four species of human hepatocellular cancer cell line (Hep-3B. Hep-G2, Huh-7 and Alexander) were cultured under humidified atmosphere of 95% air and 5% carbon dioxide, in a Dulbecco Modified Eagle culture medium (DMEM: SIGMA CHEMICAL Co., St. Louis USA) supplemented with 10% fetal bovine serum (FBS), 100 U/ml penicillin G sulphate and 100 μg/ml streptomycin sulphate.

(2) Growth Inhibition

For lecithin (other name: phosphatidylcholine (hereinafter, referred to as simply “PC”), soybean lecithin with a purity of approximately 96.5% was used, and menatetrenone and Kaytwo N (Kaytwo N: registered trade mark; manufactured by Eisai Co. Ltd.) were provided by Eisai Co. Ltd. For the study, cells (Hep-G2: 1×10⁴; others: 1×10³) in a 96-well microtiter plate were cultured one day prior to beginning the experiments. In the MTT assay method, cells in 96-well microtiter were cultured several days (2 to 4 days) in the presence of PC (1×10⁻⁸ to 1×10⁻⁴M) and menatetrenone (1×10⁻⁷ to 1×10⁻⁴ M) or Kaytwo N (registered trade mark; manufactured by Eisai Co. Ltd.) (regarding menatetrenone: 1×10⁸ to 1×10⁻⁴M). In order to evaluate the synergistic effects of menatetrenone and PC, stimulation by PC mix with menatetrenone was performed. Evaluation of the synergistic effects of menatetrenone and PC was examined by the Isobologram method (Kano et. al., Int. J. Cancer: 50, 604-610 (1992)) against the Hep-3B cell line. The survival rate was analyzed by the MTT assay method using the Tetra Color One Kit. In addition, in order to estimate the growth inhibition caused by PC, PC with a purity of approximately 99% extracted from egg yolk (SIGMA CHEMICAL Co., St. Louis USA), that is to say, egg yolk lecithin, was also examined.

(3) Statistical Analysis

Significant differences were evaluated by the Mann-Whitney U test according to StatView 5.0 (Abacus Concepts, Berkley, Calif.). Significant differences were set to P values of 0.05 or less.

### B: In Vivo Experiments

(1) Animals and Diet

Male Sprague-Dawley rats (4 weeks old) with body weights of 80 to 100 g were purchased from SLC, Inc. The rats were, three rats in each cage were held under a L/C (light-dark) cycle every 12 hours in an air-conditioned plastic cage maintained at a temperature of 23±1° C. and a relative humidity of 55±10%, and were given CE-2 basic food (Chubu Kagaku Shizai, Japan) and, freely, water. One week prior to beginning of the experiments, rats were acclimatized to such environment, and during the experiment period, they were maintained in accordance with NIH guideline referred to as Care and Use of Laboratory Animals.

(2) Experimental Design

In order to examine the chemical prevention effect of menatetrenone and PC on chemical liver cancer episode, rats were divided randomly into the eight experimental groups shown in Table 1. Note that A group, G group and H group contained six rats, and that B group, C group, D group, E group and F group contained 12 rats.

TABLE 1
Explanation of the in vivo experiment groups

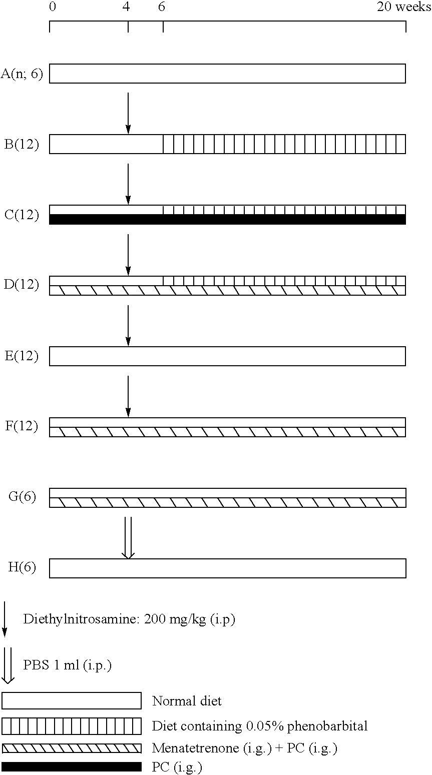

Rats in the B group, the C group and the D group, were used in a two-stage liver carcinogenesis model (refer to A Bishayee et. al., British Journal of Cancer (1995) 71, 1214-1220). Carcinogenesis was carried out by a single intraperitoneal administration (i.p.) of diethylnitrosamine (DEN: 200 mg/kg body weight) dissolved in 1 ml of phosphate-buffered physiological saline (PBS). Following a recovery period of two weeks, phenobarbital (Pb) as a promoting agent was incorporated into basic food at a proportion of 0.05% (w/w) for 14 weeks continuously thereafter. In contrast to rats in the A group that were untreated and served as controls, rats in the B group served as controls for the carcinogen (DEN-Pb). In the D group, E group and G group, menatetrenone (10 mg/day/kg body weight) and PC (16 mg/day/kg body weight) (Kaytwo N: administration via Kaytwo N) were administered to rats every two days throughout the experimental period by intragastric gavage (i.g.). As shown in Table 1, in contrast to the rats in the E group and the F group that were treated only with DEN (i.p.) as carcinogen, rats in the H group were treated with 1 ml of PBS as controls. Rats in the C group were treated only with PC (16 mg/day/kg body weight) by intragastric gavage every two days. Note that the dietary intake and body weight were measured three times per week for all the rats. Twenty weeks after the beginning of the experiment, all the rats were sacrificed by suitable diethyl anesthesia. For the last four days of experiment, incorporation of Pb into basic food was discontinued, and both ingestions, of menatetrenone and PC (administration via Kaytwo N), and PC, were discontinued. The rats were made to fast overnight prior to sacrifice by diethylether.

(3) Morphology, Histology and Histochemistry

After the rats were sacrificed, the liver was removed rapidly from each rat, the weight thereof was measured, whereafter it was observed visually. For the purpose of visual examination of nodules, 2 to 3 mm-thick sections were cut. Visual observation of nodules was carried out from two perpendicular directions, and the average diameter of each nodule was measured. Representative 1 cm-thick sections from the right posterior portion, the anterior portion and the caudate lobe of each liver were fixed in 10% buffered formalin, and used in immunohistochemical analysis by glutathione S-transferase (GST-P) and the usual hematoxylin eosin staining (H&E).

(4) GST-P Immunohistochemical Staining

In order to show GST-P-positive liver foci, liver sections were deparaffinized and the avidin-biotin-peroxidase complex (ABC) method was carried out using an anti-rat GST-P antibody (MBL Co., Ltd., Nagoya; 1:2000). In three or four slides from each liver, GST-P staining was observed with a high magnification microscope. The proportion of GST-P-positive focal surface area per surface area of small tissue fragment (%) and the number of GST-P-positive foci per unit surface area (1 cm²) were determined with Mac SCOPE Version 2.6 (Mitanishoji Co., Fukui, Japan). GST-P-positive foci comprising 10 cells or more were handled as altered hepatocellular foci.

(5) Serological Examination

Twenty weeks after the beginning of the experiment, all the rats were sacrificed by diethylether anesthesia after they were made to fast overnight. Blood samples were collected by cardiac puncture, and GPT and GOT for liver function examination, PIVKA-II as a tumor marker, and the quantity of vitamin K1 and K2 (menatetrenone) at slaughter time were examined.

(6) Statistical Analysis

Comparison of the rate of occurrence of nodules that can be visualized and the observations of GST-P-positive foci in each group, as well as comparison of serological observations in the groups treated with menatetrenone and PC or the non-treated groups, were evaluated by the Mann-Whitney U test. Significant differences were set to P values of 0.05 or less.

Experiment Results

Results of In Vitro MTT Assay Method

In all the hepatocellular cancer cell lines (Hep-3B, Hep-G2, Huh-7 and Alexander), the survival rate decreased in the presence of PC depending on the quantity added and time (refer to FIGS. 1A and 1B), and the survival rate decreased in the presence menatetrenone depending on the quantity added (refer to FIG. 1C). In addition, in the presence of Kaytwo N and of the mixture of menatetrenone and PC, compared to the experimental examples of only PC and menatetrenone, an additional effect in regard to survival rate due to PC was demonstrated (refer to FIGS. 1D and 1E). Specifically, when the case where the concentration of menatetrenone in FIG. 1C was 1×10⁻⁶ M and the result in FIG. 1D were compared, the survival of hepatocellular cancer cell line due to the addition of PC decreased, confirming an additional effect due to PC. From the above results, menatetrenone and PC each demonstrated an inhibitory effect on the multiplication of human hepatocellular cancer cell lines.

FIG. 2 is a result showing the evaluation of the synergistic effects of menatetrenone and PC on the Hep-3B cell line, in the present invention, by the Isobologram method (Kano et. al., Int. J. Cancer: 50, 604-610 (1992)). FIG. 2A shows the results for menatetrenone (represented as VK in FIG. 2A) alone on the growth inhibition of the Hep-3B cell line, which are results in which the values of the concentration of menatetrenone added were represented as the horizontal axis, normalized with the concentration of menatetrenone at which there was an inhibitory effect of 50% as the criterion, and the logarithm of the ratio of growth inhibition as the vertical axis. Note that the actual concentrations of menatetrenone were 0.32×10⁻⁵ M, 0.63×10⁻⁵ M, 1.25×10⁻⁵ M, 2.5×10⁻⁵ M and 5.0×10⁻⁵ M. Meanwhile, FIG. 2B shows the results for PC alone on the growth inhibition of Hep-3B cell line, which are results in which the values of the concentration of PC added were represented as the horizontal axis, normalized with the concentration of PC at which there was an inhibitory effect of 50% as the criterion, and the logarithm of the ratio of growth inhibition as the vertical axis. Note that the actual concentrations of PC were 0.32×10⁻⁵ M, 0.63×10⁻⁵ M, 1.25×10⁻⁵ M, 2.5×10⁻⁵ M and 5.0×10⁻⁵ M.

Next, FIG. 2C shows the results of growth inhibition of the Hep-3B cell line by the addition of menatetrenone only, and the addition of a mixture of menatetrenone and PC. From the results shown in FIG. 2C, the growth inhibitory effect is greater for the mixture with PC added further, rather than menatetrenone alone, and it is clear that when the concentration of PC added further is high, the growth inhibitory effect is greater. Note that in FIG. 2C, dose-response curves are shown when the quantity of menatetrenone added was varied with concentrations in PC of 0.32×10⁻⁵ M and 0.63×10⁻⁵ M.

From the above results, as shown in FIG. 2D, in order to evaluate the effects of menatetrenone and PC on the growth inhibition of Hep-3B cell line, Mode I line and Mode II line were created according to the Isobologram method (Kano et. al., Int. J. Cancer: 50, 604-610 (1992)), and the supra-additive region, the envelop of additivity region and the sub additive region were determined. Then, when the dose-response curve due to menatetrenone and PC obtained in FIG. 2C was used, the data obtained in FIG. 2C were positioned at [triangle] and [cross] represented in FIG. 2D. This position belongs to the supra-additive region in the Isobologram method analysis, illustrating the existence of synergistic effect on the growth inhibition of Hep-3B cell line by menatetrenone, due to PC addition.

From the above results, an additional effect was demonstrated on the growth inhibition of hepatocellular cancer cell line for Kaytwo N comprising soybean lecithin as PC added to menatetrenone, and the mixture of menatetrenone and PC was shown to have synergistic effects on the growth inhibition of hepatocellular cancer cell line from the analysis result of the Isobologram method.

In Vivo Experiment Results

(1) On Deaths

Since three rats died within two weeks after DEN (i.p.), two in the B group and one in the C group, they were not used in the experiments of the present invention. However, no death of rats from other groups was observed during the experiment.

(2) On the Body Weight and Liver Weight

In the B group, the C group, the D group, the E group and the F group, accompanying a decrease in dietary intake, the body weight of the rats decreased during the two weeks after DEN (i.p.). However, no significant difference was observed for the final body weight among all eight groups. The body weight of rats in the C group, the D group, the F group and the G group were one that was comparable to the body weight of rats in the A group, suggesting that in the present experiment, the addition of Kaytwo N or PC is not one that exerts a particularly negative influence on the development response of the rats. The average weight of livers from rats in the B group, the C group and the D group who ingested diet containing Pb, and the average liver weight ratios of the foregoing (that is to say, liver with respect to body weight), were heavier than other rats. Specifically, average weight of liver from rats in the B group, the C group and the D group was 18.9 g (average in other groups: 16.7 g), and the relative average liver weight ratio was 0.0347 (average in other groups: 0.0301). Note that Pb, a strong mitogen, has the capability of enhancing cell cycles within enzyme activity altered foci, which provokes hypertrophy and/or hyperplasia (Chong-Kuei Lii, et. al., Nutrition and Cancer 38 (1), pp 50-59, 2000; Meenakshi Vijayaraghavan, et al., Jpn. J. Cancer Res; 91, 780-785, August 2000).

(3) Effects of Kaytwo N and PC on Nodule Development

In the hepatocyte nodules in the E group, F group and H group, as well as the hepatocyte nodules in the control (A group) and Kaytwo N control group (G group), the nodules were not observed by visual observation. However, hepatocyte nodules having a grayish white surface were observed in several rats in the B group, the C group and the D group, in particular, in the rat liver of the B group, tumors of 5 mm maximum diameter were histologically assumed to be precancerous nodules (refer to FIGS. 3A and 3B). Comparing to the DEN-Pb control (B group), the rate of incidence of visible nodules decreased significantly in the C group and D group. The rate of incidence of nodules in the C group was higher of that in the D group. This is because Kaytwo N comprising PC (D group) demonstrated an additional growth inhibition effect (refer to FIG. 3C).

(4) Effects of Kaytwo N and PC on Liver Histology

With the livers in the B group, the C group and the D group, it was found that morphologically altered hepatocyte groups were scattered; however, such scattering was not observed in the non-treated control group (A group), Kaytwo N control group (G group) and PBS (i.p.) (H group). In the B group, the C group and the D group, clear cell foci, fatty liver alteration and necrotic foci where inflammatory cells are present that are clearly identifiable from the surrounding normal interstitial tissue, were found as focal alterations in H&E stained fragments on liver slides.

(5) Effects of Kaytwo N and PC on the Induction of GST-P-Positive Foci

The liver of rats in the normal control group (A group), the Kaytwo N control group (G group) and PBS (i.p.) (H group) were found to be normal from the histological point of view; however, GST-P-positive staining was observed in small foci. Meanwhile, GST-P-positive foci where morphologically altered cells were present expanded in the B group, the C group and the D group (refer to FIG. 4C and FIG. 4D). In the C group and the D group, when PC and Kaytwo N were added, respectively, expansion of GST-P-positive foci was attenuated significantly compared to the B group. The GST-P-positive foci expanded more in the C group than the D group (refer to FIGS. 5A and 5B).

Due to the addition of PC and Kaytwo N, a remarkable diminution of the rate of incidence of visible nodules was effected, along with a diminution of the number and surface area of GST-P-positive liver foci in the promotion by Pb of the DEN carcinogenesis initiation action. Note that the opinion that nodules are precursors of liver cancer is supported by many observations (refer to A Bishayee et, al., British Journal of Cancer (1995) 71, 1214-1220).

It is generally accepted that a GST-P-positive focus is an identifiable evidence of the occurrence of a tumor at an early stage (refer to R. Schulte-Hermann et. al., Carcinogenesis Vol. 7 No. 10 pp. 1651-1655 (1986); A Bishayee et. al., British Journal of Cancer (1995) 71, 1214-1220; Thomas S. Winokur et. al., Carcinogenesis Vo. 11 No. 3 pp. 365-369 (1990)). In addition, a GST-P-positive focus is assumed to be a precancerous lesion (refer M. C. Carrillo et al., Experimental Gerontology 36 (2001) pp 255-265; R. Schulte-Hermann et al., Carcinogenesis Vol. 7 No. 10 pp 1651-1655 (1986); Yulia Y. Maxuitenko et al., Carcinogenesis Vo. 14. No. 11 pp. 2423-2425 (1993)). The above results clearly show the role of Kaytwo N and PC in the number of GST-P-positive precancerous lesions in a unit surface area (cm2) of a rat liver started by DEN-Pb (refer to British Journal of Cancer (1995) 71). As a GST-P-positive focus is a transition stage into a malignant tumor, the capabilities of Kaytwo N and PC to attenuate the enlargement of a GST-P-positive focus exert a significant influence on the early stage of liver cancer development by having PC and/or menatetrenone blocking the cells that have received DEN-Pb carcinogenesis initiation action from developing into precancerous foci, through alteration of efficiency of carcinogenesis due to DEN-Pb.

(6) Serological Examination

Significant contribution of menatetrenone due to Kaytwo N addition was shown in the D group, the F group and the G group compared to the A group, the B group and the E group. Meanwhile, no significant difference was observed among each of the groups for vitamin K1 (refer FIGS. 6A and 6B), The tumor marker (PIVKA-II) and transaminase (GPT and GOT) in the groups where Kaytwo N was added (D group, F group and G group) were significantly suppressed (refer to FIGS. 6C, 6D and 6E) compared to the values in the groups where Kaytwo N was not added (A group, B group and E group). Deterioration in the serological examination, specifically, deterioration of GPT, GOT and PIVKA-II, was suppressed by the addition of Kaytwo N.

### INDUSTRIAL APPLICABILITY

According to the present invention, growth inhibition of liver cancer by menatetrenone is promoted by combination with a lecithin as phospholipid compared to when using vitamin K alone, providing a pharmaceutical composition as a better liver cancer treatment or prevention agent.

### BRIEF DESCRIPTION OF THE DRAWINGS

FIG. 1A shows the results of the variation in the number of surviving cells when, with respect to each liver cancer cell strain, a culture was carried out for three days in the presence of various concentrations (1×10⁻⁸ to 1×10⁻⁴ M) of PC. FIG. 1B shows the results of the variation in the number of surviving cells when, with respect to each liver cancer cell strain, culture was carried out for several days by fixing the concentration of PC (1×10⁻⁵). FIG. 10 shows the results of the variation in the number of surviving cells when, with respect to each liver cancer cell strain, a culture was carried out for three days in the presence of various concentrations (1×10⁻⁷ to 1×10⁻⁴ M) of menatetrenone. FIG. 1D shows the results of the variation in the number of surviving cells when, with respect to each liver cancer cell strain, a culture was carried out for three days in the presence of menatetrenone (1×10⁻⁶ M) and various concentrations (1×10⁻⁸ to 1×10⁻⁶ M) of PC. FIG. 1E is a result showing the average of the number of surviving cells when, with respect to each liver cancer cell strain, a culture was carried out for three days in the presence of menatetrenone (1×10⁻⁷ to 1×10⁻⁴ M) and Kaytwo N. Note that Kaytwo N comprises not only menatetrenone, but also soybean lecithin;

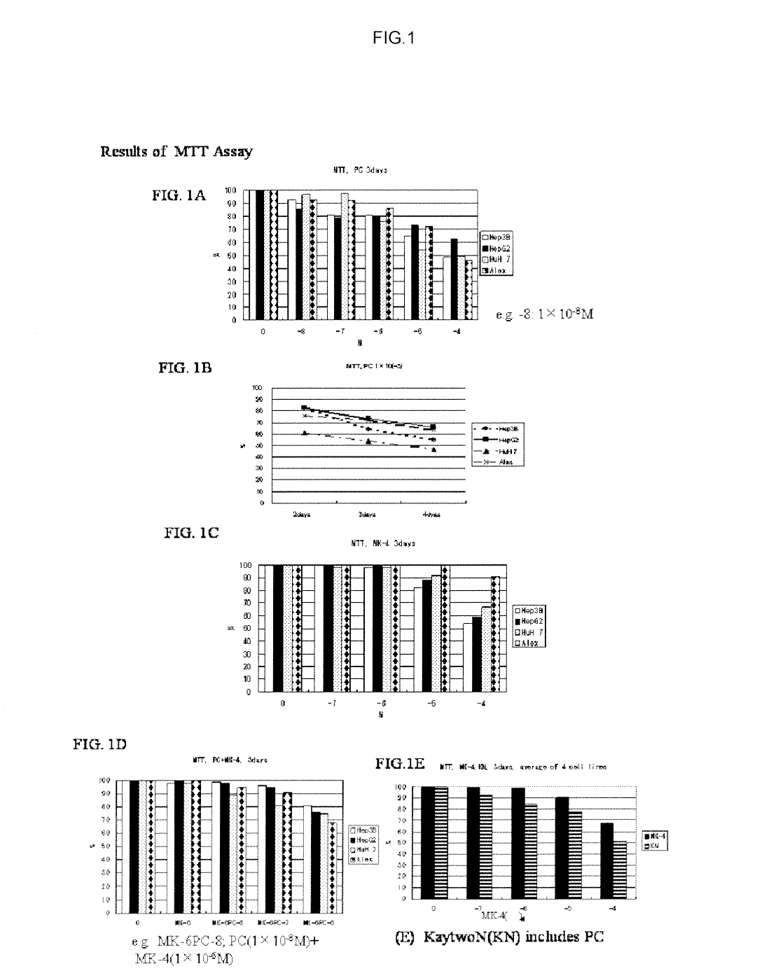

FIG. 2A shows the results for menatetrenone alone on the growth inhibition of the Hep-3B cell line, which are results in which the values of the concentration of menatetrenone added were represented as the horizontal axis, normalized with the concentration of menatetrenone at which there was an inhibitory effect of 50% as the criterion, and the logarithm of the ratio of growth inhibition as the vertical axis. Note that VK in the present figure means menatetrenone. FIG. 2B shows the results for PC alone on the growth inhibition of Hep-3B cell line, which are results in which the values of the concentration of PC added were represented as the horizontal axis, normalized with the concentration of PC at which there was an inhibitory effect of 50% as the criterion, and the logarithm of the ratio of growth inhibition as the vertical axis. FIG. 2C shows the results of growth inhibition of the Hep-3B cell line by the addition of menatetrenone only, and the addition of a mixture of menatetrenone and PC. FIG. 2D is a figure describing the effects of menatetrenone and PC on the growth inhibition of the Hep-3B cell line according to the Isobologram method (Kano et. al., Int. J. Cancer: 50, 604-610 (1992));

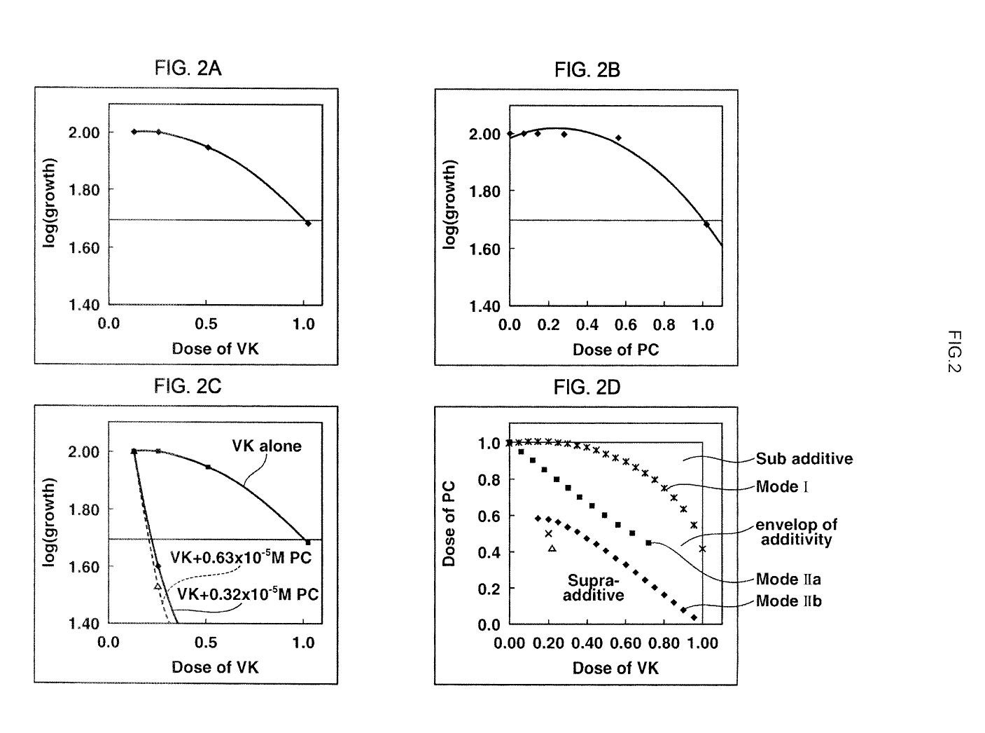

FIG. 3 shows the results in relation to visual observation of nodules according to present invention. FIG. 3A shows a visual observation, FIG. 3B shows a microscope observation of nodules, and FIG. 30 shows the results for the number of visible nodules per rat liver;

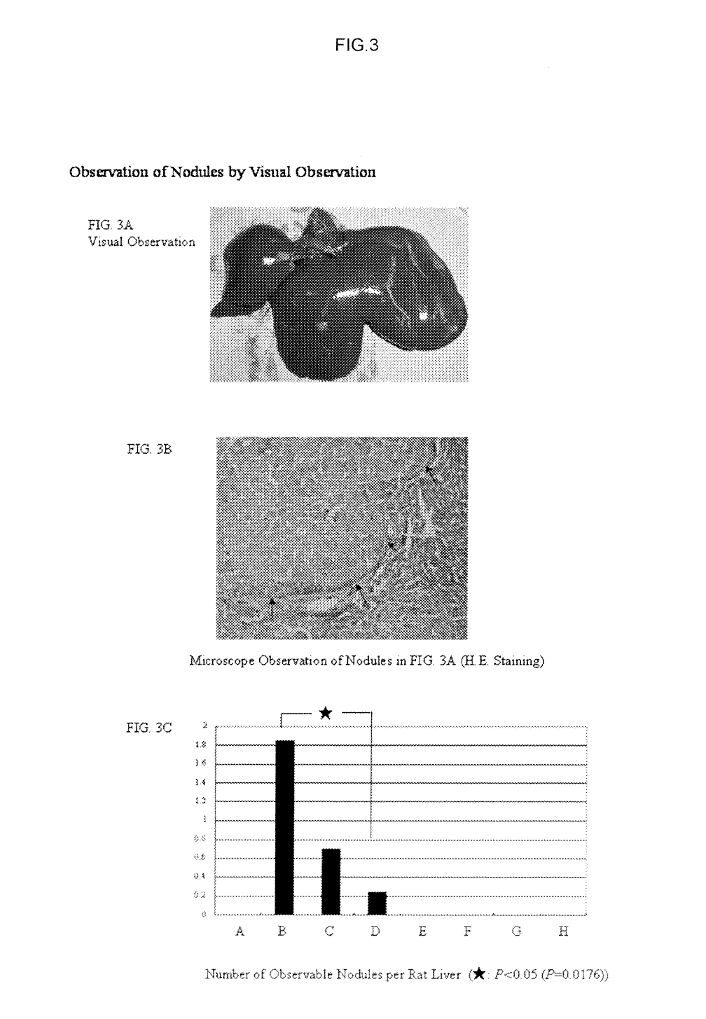

FIG. 4 shows the results in relation to histological observations obtained in experiments according to the present invention. FIG. 4A shows the result of histological observation in the A group, FIG. 4B shows the result of histological observation where clear cell foci and fatty degeneration are present in the B group, FIG. 4C shows a clear cell foci within the sample in the B group, and FIG. 4D shows the result of GST-P positive staining of clear cell foci in serial sections of FIG. 4C;

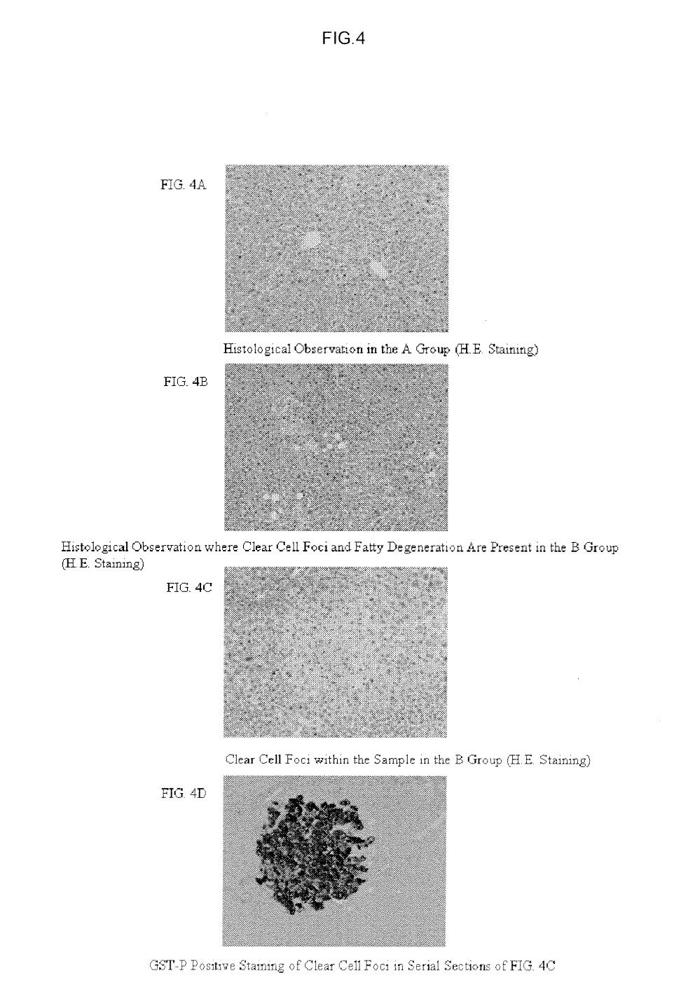

FIG. 5 shows the results obtained in immunohistochemical observations in the experiments according to the present invention. FIG. 5A shows the results of surface area ratio of GST-P-positive staining in each group, and FIG. 5B shows the result of the number of foci where GST-P-positive staining is present/cm2 in each group; and

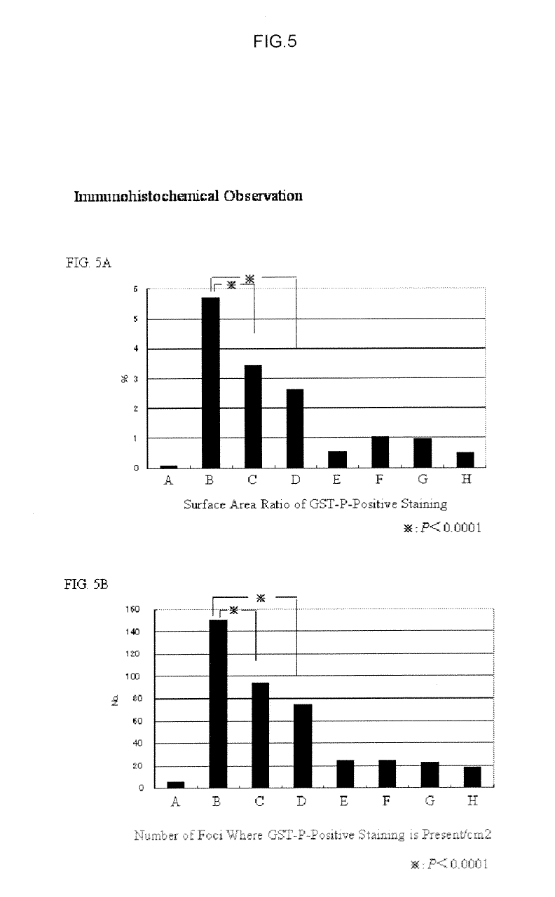

FIG. 6 shows the results in relation to serological observations obtained in the experiments according to the present invention. FIG. 6A shows the results of the quantity of vitamin K2 in non-addition/addition of Kaytwo N (KN−/KN+), FIG. 6B shows results of the quantity of vitamin K1 in non-addition/addition of Kaytwo N (KN−/KN+), FIG. 6C shows results of the quantity of PIVKA-II in non-addition/addition of Kaytwo N (KN−/KN+), and FIGS. 6 (D) and 6 (E) show results of the quantity of GPT and GOT in non-addition/addition of Kaytwo N (KN−/KN+).

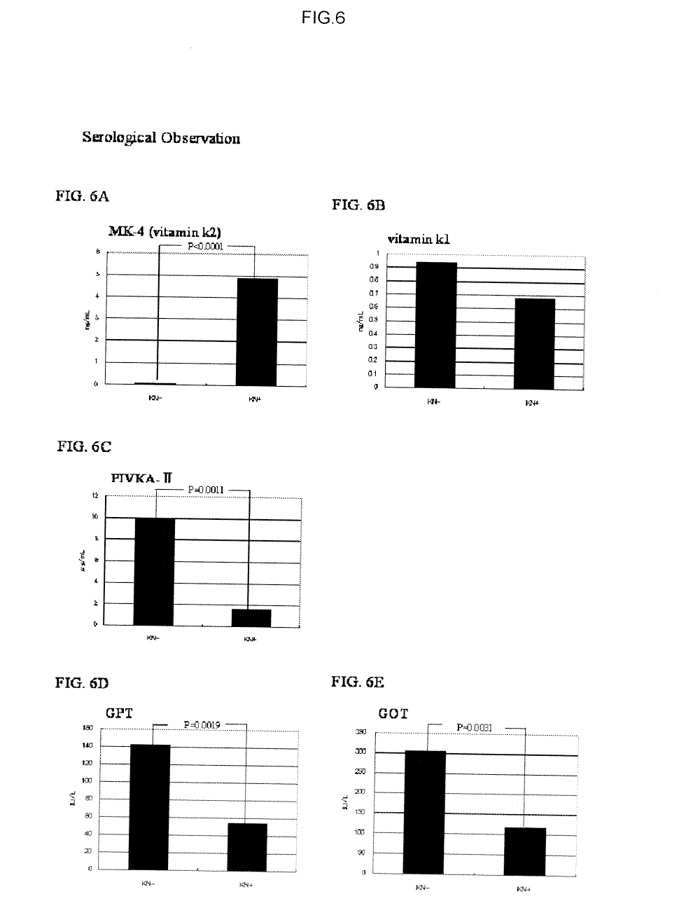

1. A pharmaceutical composition for treating or preventing liver cancer, comprising menatetrenone and a phospholipid, wherein the phospholipid is egg yolk lecithin or soybean lecithin.

2-4. (canceled)

5. The pharmaceutical composition according to claim 1, wherein a mixing ratio of said menatetrenone and said phospholipid (menatetrenone/phospholipid) is a ratio in mg/day/body weight of from 1/10 to 10/1.

6. The pharmaceutical composition according to claim 1, wherein said menatetrenone is administered with a dose of from 1.0 to 100 mg/day/body weight.

7. The pharmaceutical composition according to claim 1, wherein said phospholipid is administered with a dose of from 1.0 to 100 mg/day/body weight.

8. The pharmaceutical composition according to claim 1, which is administered orally.
